(19) United States
(12) Patent Application Publication
(10) Pub. No.: US 2007/0135375 A1
(43) Pub. Date: Jun. 14, 2007

(54) Sulfamoyl sulfonate prodrugs

(76) Inventors: Ralf Wyrwa, Rothenstein (DE); Reinhard Nubbemeyer, Berlin (DE); Ursula Ganzer, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/605,473

(22) Filed: Nov. 29, 2006

Related U.S. Application Data
(60) Provisional application No. 60/742,526, filed on Dec. 6, 2005.

(30) Foreign Application Priority Data
Nov. 30, 2005 (DE) 10 2005 057 408.4

(51) Int. Cl. A61K 31/7072; A61K 31/56; A61K 31/4745; C07H 19/12; C07D 453/04; C07J 1/00

(52) U.S. Cl. 514/49; 514/182; 514/305; 552/626; 546/136; 536/28.3

(57) ABSTRACT

The invention relates to sulfamoyl sulfonate prodrugs of general formula I, 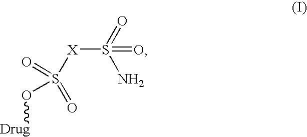 a process for their production, pharmaceutical compositions that contain these compounds, and their use for the production of orally available pharmaceutical agents. The compounds according to the invention bind to carbonic anhydrases and inhibit these enzymes.

This application claims the benefit of the filing date of U.S. Provisional Application Ser. No. 60/742,526 filed Dec. 6, 2005.

The invention relates to sulfamoyl sulfonate prodrugs of general formula I, 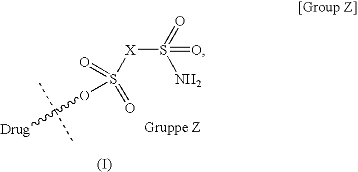
a process for the production of these prodrugs, pharmaceutical compositions that contain these compounds, and their use for the production of orally available pharmaceutical agents.

From WO 01/91797, steroidal compounds that are bonded via an —SO₂NR¹R² group to erythrocytes and that accumulate there are known. The concentration ratio of the compounds between erythrocytes and plasma is 10-1000:1, preferably 30-1000:1, so that a depot formation in the erythrocytes can be mentioned. Because of the strong binding of the compounds to the erythrocytes, metabolization is avoided during passage through the liver. Disadvantageously, no therapy-relevant active ingredient levels are given despite a reduced metabolization with the indicated dosages. Reasons for this can be sought in excessive binding to erythrocytes, an enzyme-induced cleavage and in low solubilities.

It is the object of the invention to provide new prodrugs that are orally available and that, in comparison to the prior art, ensure a therapy-relevant active ingredient level even at a low dosage.

This object is achieved by sulfamoyl sulfonate prodrugs of general formula (I), in which a sulfamoyl radical is bonded to the drug that is to be released via a spacer X by means of a sulfonate bond.

Sulfamoyl sulfonate prodrugs of general formula (I) are 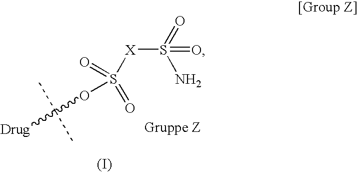
in which
X is a C₁₋₁₂-alkanediyl-, a CₚF₂ₚ group where p=1-5, a C₃₋₈-cycloalkanediyl-, an arylene-, a heteroalkanediyl-, a C₁₋₄-alkanediylaryl-, a C₁₋₄-alkanediyl-C₃₋₈-cycloalkyl- or a C₃₋₈-cycloalkanediyl-C₁₋₄-alkyl group, and
Drug is a pharmaceutical active ingredient that can form a sulfonate via an OH group, such as steroids, anti-malaria agents, nucleosides, or isoflavonoids, which can optionally be substituted.

The sulfamoyl sulfonate compounds according to the invention bond to erythrocytes, are readily water-soluble and are hydrolytically cleaved without assistance from enzymes.

For the purpose of this invention, “C₁₋₁₂alkanediyl group” is defined as a double-bonded, branched or straight-chain alkylene radical with up to 12 carbon atoms that optionally can be substituted with, e.g., halogen atoms, hydroxy groups, or nitrile groups. As examples, a methane-1,1-diyl-, ethane-1,2-diyl, propane-1,3-diyl-, butane-1,4-diyl-, pentane-1,5-diyl-, hexane-1,6-diyl-, octane-1,8-diyl-, and undecane-1,11-diyl group can be mentioned.

For the purpose of this invention, CₚF₂ₚ group with p=1-5 is defined as a branched or straight-chain perfluorinated alkyl radical with up to 5 carbon atoms. As examples, a perfluoropropane-1,3-diyl-, perfluorobutane-1,4-diyl-, and perfluoropentane-1,5-diyl group can be mentioned.

The above-mentioned “C₃₋₈-cycloalkanediyl group” means, according to the invention, a double-bonded, mono- or bicyclic, carbocyclic group with 3 to 8 carbon atoms that optionally can be substituted with halogen atoms, hydroxy groups, and nitrile groups, such as, for example, with a cyclobutane-1,3-diyl-, cyclopentane-1,3-diyl- or a cyclohexane-1,4-diyl group.

The above-mentioned “arylene group” means, according to the invention, a double-bonded, aromatic mono- to tricyclic, carbocyclic group with 6 to 15 carbon atoms that optionally can be substituted with halogen atoms, hydroxy groups, nitrile groups, and alkyl groups, such as, for example, with an m-phenylene-, p-phenylene-, phenanthrylene- or a naphthalene group.

The heteroarylene radical includes, in each case, 5-16 ring atoms and contains, instead of carbon, one or more heteroatoms that are the same or different, such as oxygen, nitrogen or sulfur, in the ring. The heteroaryl radical can be mono-, bi- or tricyclic.

For example, there can be mentioned: thienyl, furanyl, pyrrolyl, oxazolyl, thiazolyl, imidazolyl, pyrazolyl, isoxazolyl, isothiazolyl, oxadiazolyl, triazolyl, thiadiazolyl, benzofuranyl, benzothienyl, benzothiazole, benzoxazolyl, benzimidazolyl, indazolyl, indolyl, isoindolyl, pyridyl, pyridazinyl, pyrimidinyl, pyrazinyl, triazinyl, quinolyl, and isoquinolyl.

For the purpose of the invention, a heteroalkanediyl group is a double-bonded, straight-chain or branched, saturated or unsaturated heteroalkyl radical with, in each case, 1-6 carbon atoms and can contain, instead of carbon, one or more heteroatoms that are the same or different, such as oxygen, nitrogen or sulfur, such as, for example, a bis-ethylenoxy radical.

The “C₁₋₄-arylalkanediyl group” is an aryl group that is bonded to a skeleton via a C₁-C₄-alkanediyl group, whereby the alkanediyl group can be straight-chain or branched. For example, benzyl or phenethylene can be mentioned.

The “C₃₋₈-cycloalkyl-C₁₋₄-alkanediyl group” means, for example, cycloalkyl-(CH₂)—, cycloalkyl-(C₂H₄)—, cycloalkyl-(C₃H₆)—, cycloalkyl-(C₄H₈)—, or cycloalkyl-(C₅H₁₀)—. In this case, cycloalkyl can be cyclopropyl, cyclobutyl, cyclopentyl, cyclohexyl, cycloheptyl or cyclooctyl.

“C₁₋₄-Alkyl-C₃₋₈-cycloalkanediyl group” is defined as methylcycloalkanediyl, ethyl-cycloalkanediyl, propylcycloalkanediyl, butylcycloalkanediyl, or pentylcycloalkanediyl. In this case, cycloalkanediyl can be cyclopropane-1,3-diyl, cyclobutane-1,4-diyl, cyclopentane-1,5-diyl, cyclohexane-1,6-diyl, cycloheptane-1,7-diyl or cyclooctane-1,8-diyl.

In the context of this invention, the term “halogen atom” is defined as a fluorine, chlorine, bromine or iodine atom, preferably a fluorine, chlorine, or bromine atom.

A pharmaceutical active ingredient that can form a sulfonate via an OH group means, for the purpose of the invention, the following:
Steroids: Estrogens, for example estradiol or estriol, or androgens, for example testosterone, MENT (7α-methyl-19-nortestosterone), eF-MENT (11-fluoro-7α-methyl-19-nortestosterone), nandrolone, DHT (dihydrotestosterone), or gestagens, for example norethisterone, dienogest or levonorgestrel, or corticoids, for example cortisol
Anti-malaria agents: quinine, cinchonidine, hydroxychloroquine, primaquine, mefloquine; or
Nucleosides: consisting of a sugar, such as ribose or deoxyribose, and a base, such as adenine, guanine, cytosine, thymine or uracil, and also zidovudine, brivudine, indinavir, and nelfinavir;
Isoflavonoids: genisteine.

Especially preferred compounds are specified as follows:
1) 3-hydroxyestra-1,3,5(10)-trien-17β-yl 3′-sulfamoylphenyl sulfonate,
2) 3-acetoxyestra-1,3,5(10)-trien-17β-yl 3′-sulfamoylphenyl sulfonate,
3) 3-tert-butyldimethylsilyloxyestra-1,3,5(10)-trien-17β-yl 3′-sulfamoylphenyl sulfonate,
4) 3-hydroxyestra-1,3,5(10)-trien-17β-yl 4′-sulfamoylphenyl sulfonate,
5) 2-methoxy-3-hydroxyestra-1,3,5(10)-trien-17β-yl 3′-sulfamoylphenyl sulfonate,
6) 3,16α-dihydroxyestra-1,3,5(10)-trien-17β-yl 3′-sulfamoylphenyl sulfonate,
7) 3,17β-dihydroxyestra-1,3,5(10)-trien-16α-yl 3′-sulfamoylphenyl sulfonate,
8) 3-benzoyloxyestra-1,3,5(10)-trien-17β-yl 3′-sulfamoylphenyl sulfonate,
9) quinine-3′-sulfamoylphenyl sulfonate,
10) cinchonidine-3′-sulfamoylphenyl sulfonate,
11) zidovudine-3′-sulfamoylphenyl sulfonate,
12) 3-oxoandrost-4-en-17β-yl 3′-sulfamoylphenyl sulfonate,
13) 3-oxoandrostan-17β-yl 3′-sulfamoylphenyl sulfonate,
14) 3-oxo-7α-methylandrost-4-en-17β-yl 3′-sulfamoylphenyl sulfonate,
15) 3-oxoestr-4-en-17β-yl 3′-sulfamoylphenyl sulfonate, and
16) brivudine-3′-sulfamoylphenyl sulfonate.

The therapeutically relevant drug compound is released through hydrolysis from the compounds according to the invention.

In-Vitro Tests:

Carbonic Anhydrase Inhibition

Test Principle:

Photometric determination of the inhibition of human carbonic anhydrase I or II with sulfonamides or sulfamates on microtiter plates with the aid of enzymatic conversion of nitrophenyl acetate with a color change from colorless to yellow.

TABLE 1
IC₅₀ Inhibiting Values of Human Carbonic Anhydrase I

| | CA II | CA I |
|---|---|---|
| Inhibitor | IC₅₀(nM) | IC₅₀ (nM) |
| Estradiol-3-sulfamat | 34 | 157 ± 10.6 |
| 3-Oxoandrost-4-en-17β-yl | 120 | 2300 |
| 3′-aminosulfonylphenyl- | | |
| sulfonat | | |
| 3-Oxo-7α-methylandrost-4- | 120 | 2300 |
| en-17β-yl 3′-aminosulfonyl- | | |
| phenylsulfonat | | |
| 3-Hydroxyestra-1,3,5(10)- | 81 | 1700 |
| trien-17β-yl 3′-aminosulfo- | | |
| nylphenylsulfonat | | |
| Zidovudinsulfonat | 1100 | 2900 |
| Acetazolamid | 61 | 1200 |
| (bekannter CA-Hemmer) | | 1900 ¹⁾ |

Literature: ¹⁾C. Landolfi, M. Marchetti, G. Ciocci, and C. Milanese, Journal of Pharmacological and Toxicological Methods 38, 169-172 (1997).
[Key to Table 1:]
Estradiol-3-sulfamat = Estradiol-3-sulfamate
3-Oxoandrost-4-en-17β-yl 3′-aminosulfamoylphenylsulfonat = 3-Oxoandrost-4-en-17β-yl 3′-sulfamoylphenyl sulfonate
3-Oxo-7α-methylandrost-4-en-17β-yl 3′-aminosulfamoylphenylsulfonat = 3-Oxo-7α-methylandrost-4-en-17β-yl 3′-aminosulfamoylphenyl sulfonate
3-Hydroxyestra-1,3,5(10)-trien-17β-yl 3′-aminosulfonylphenylsulfonat = 3-Hydroxyestra-1,3,5(10)-trien-17β-yl 3′-aminosulfonylphenyl sulfonate
Zidovudinsulfonat = Zidovudine Sulfonate
Acetazolamid (bekannter CA-Hemmer) = Acetazolamide (Known CA Inhibitor)

It was found that the sulfamoyl sulfonate prodrugs according to the invention surprisingly readily inhibit the carbonic anhydrase II. A concentration of the prodrugs, according to the invention, in the erythrocytes can be deduced from this.

Physicochemical Data

Solubility in Water

a) Kinetic Measurement:

The compounds according to the invention were measured as 10 mmol DMSO solution in a 0.01 M phosphate buffer solution at pH 7.4 and 25° C. with nephelometry and turbidity.

While in a turbid state, the solution to be tested was added drop by drop to the buffer solution until a precipitate settled.

The precipitate was detected in a dilution series (compounds in a phosphate buffer solution according to the invention) by nephelometry.

b.) Thermodynamic Measurement:

The compounds in solid form according to the invention were added to an excess of an aqueous buffer system of various pH values. It was stirred 24 hours at 25° C. After centrifuging, the solution was examined with HPLC (HPLC: column: Xterra MS C18 2.5 μm, 30×4.6 mm). Two standard gradient systems were used based on the compounds to be measured:
Acidic gradient: A: water/0.01% trifluoroacetic acid, B: acetonitrile/0.01% trifluoroacetic acid—0 min 5% B, 0-3 min 65% B, 3-5 min 65% B, 5-6 min 5% B

Alkaline gradient: A: water/0.025% ammonia, B: acetonitrile/0.025% ammonia—0 min 20% B, 0-3 min 80% B, 3-5 min 80% B, 5-6 min 20% B.

TABLE 2
Water Solubility

| Substance | Solubility in Water | |
|---|---|---|
| Estradiol-3-sulfamate | 0.15 mg/100 ml | |
| Testosterone Propionate | insoluble | |
| 3-Hydroxyestra-1,3,5(10)-trien-17β-yl 3′- | 3 | mg/l |
| sulfamoylphenyl Sulfonate | | |
| 3-Oxoandrost-4-en-17β-yl 3′-sulfamoyl- | 5 | mg/l |
| phenyl Sulfonate | | |
| 3-Oxo-7α-methylandrost-4-en-17β-yl 3′- | About 6 | mg/l |
| sulfamoylphenyl Sulfonate | | |

The compounds according to the invention show a higher solubility when compared to sulfamate- and carboxylic acid ester prodrugs, which allows for better absorption in the intestine.

Hydrolysis

The compounds according to the invention were measured as a DMSO solution in an aqueous buffer of various pH values at 37° C.

The quantification took place through HPLC (HPLC column: Xterra MS C18 2.5 μm 4.6×30 mm). Based on the test substances to be measured, the following gradient system was used for the HPLC:
Acidic gradient: A: water/0.01% trifluoroacetic acid, B: acetonitrile/0.01% trifluoroacetic acid—0 min 5% B, 0-3 min 65% B, 3-5 min 65% B, 5-6 min 5% B
Alkaline gradient: A: water/0.025% ammonia, B: acetonitrile/0.025% ammonia—0 min 20% B, 0-3 min 80% B, 3-5 min 80% B, 5-6 min 20% B. The quantification took place after 1 and 2 hours and after 24 hours.
   Stability in Simulated Gastric Juice:

Solutions of the compounds according to the invention were incubated at 37° C. in simulated gastric juice (aqueous NaCl solution with pepsin, pH˜1.2).

The quantification took place through HPLC (HPLC-column: Xterra MS C18 2.5 μm 4.6×30 mm) using the gradient system:

A: water/0.01% trifluoroacetic acid, B: acetonitrile/0.01% trifluoroacetic acid—0 min 5% B, 0-3 min 65% B, 3-5 min 65% B, 5-6 min 5% B.

The quantification took place after 0.5, 1, 1.5 and 2 hours.

TABLE 3
Hydrolysis/Stability in Gastric Juice

| | Decomposition in % | | | | | | |
|---|---|---|---|---|---|---|---|
| | pH = 1, 37° C. | | | | pH = 7.4, 37° C. | | |
| Substance | 30 min | 1 h | 2 h | 24 h | 1 h | 2 h | 24 h |
| 3-Hydroxyestra-1,3,5 | 4 | 7 | 14 | 80 | 6 | 13 | 84 |
| (10)-trien-17β-yl 3′- | (pH = 1.2) | | | | | | |
| sulfamoyl-phenyl | | | | | | | |
| sulfonate | | | | | | | |
| 3-Oxoandrost-4-en- | 3 | 6 | 11 | 78 | 6 | 12 | 75 |
| 17β-yl 3′-sulfamoyl- | (pH = 1.2) | | | | | | |
| phenyl sulfonate | | | | | | | |
| 3-Oxo-7α-methyl- | | 6 | 11 | 71 | 6 | 11 | 70 |
| androst-4-en-17β-yl | | | | | | | |
| 3′-sulfamoylphenyl | | | | | | | |
| sulfonate | | | | | | | |
| 3-Oxoandrost-4-en- | 0 | 0 | 0 | 0 | 0 | 0 | 0 |
| 17β-yl n-propionate | | | | | | | |

Carboxylic acid esters are relatively stable in gastric juice (pH˜1) and in the intestine (pH˜7.4), but are cleaved when passing through the intestine by esterases that exist there. During passage through the stomach, however, the stable prodrug remains almost complete.

The cleavage of the carboxylic acid ester thus takes place when passing through the intestine and in the liver.

No enzymes (esterases) are known for sulfonic acid ester. It was therefore surprising that the sulfonates are still cleaved, whereby a simple hydrolysis takes place. Although a slow hydrolysis already takes place in the gastric juice and when the pH=7.4, the sulfonates have adequate stability to pass through the stomach and the intestine. An esterase cleavage does not take place in the intestine wall. The first-pass effect in the liver is avoided since the sulfonates are bonded with the sulfonamide group to the erythrocytes.

The compounds of general formula (I) according to the invention can, as a function of the meaning of “drug,” be used for the treatment and/or prophylaxis of various clinical pictures. For example, the compounds of general formula (I) can be used if the “drug” is a steroid, such as androgen or estrogen, in hormone replacement therapy (HRT) in women and men, or in the treatment of hormonally-induced diseases in men (prostrate cancer, breast cancer, hypogonadism) and in women (endometriosis, breast cancer). In addition, the compounds of general formula (I) according to the invention, in which “drug,” for example, stands for an androgen or estrogen, can be used for birth control in men and women.

The use of additional active ingredients mentioned for “drug,” such as quinine, cinchonidine, hydroxychloroquine, primaquine or mefloquine, relates to the treatment of malaria.

Compounds of general formula (I) according to the invention, in which “drug” means a cortisol derivative, can be used for the treatment and prophylaxis of inflammatory and/or allergic diseases that are influenced by immunosuppressive agents and/or antiproliferative agents.

Prodrugs according to the invention, in which “drug” means a nucleoside (zidovudine, brivudine, indinavir, nelfinavir), can be used for the treatment of viral diseases (herpes, HIV).

In addition, subjects of the invention are the pharmaceutical compositions that contain the compounds of general formula (I) according to the invention and optionally additional active ingredients, for example gestagens (norethisterone, dienogest, drospirenone, levonorgestrel), antigestagens (mifepristone, onapristone) and/or progesterone receptor modulators (mesoprogestins, such as asoprisnil).

These pharmaceutical compositions and pharmaceutical agents are preferably administered orally. In addition to the usual vehicles and/or diluents, they contain at least one compound of general formula I.

Dosage

The prodrugs according to the invention can be administered orally.

Generally, satisfactory results are to be expected both for the treatment and/or prophylaxis of the above-mentioned indications or for birth control if the dosage is carried out in such a way that after the administration of the prodrugs, an amount of the corresponding active ingredient (“drug”) is released that corresponds to the highest pharmaceutically used maximum dose of the respective “drug” substance.

The pharmaceutical agents of the invention are produced in a known way with the usual solid or liquid vehicles or diluents and the commonly used pharmaceutical-technical adjuvants corresponding to the desired form of administration with a suitable dosage. The preferred preparations are in a dispensing form that is suitable for oral administration. Such dispensing forms are, for example, tablets, film tablets, coated tablets, capsules, pills, powders, solutions or suspensions or else depot forms.

Corresponding tablets can be obtained by, for example, mixing the active ingredient with known adjuvants, for example inert diluents such as dextrose, sugar, sorbitol, mannitol, polyvinylpyrrolidone, explosives such as corn starch or alginic acid, binding agents such as starch or gelatin, lubricants such as magnesium stearate or talc, and/or agents for achieving a depot effect, such as carboxylpolymethylene, carboxylmethyl cellulose, cellulose acetate phthalate or polyvinyl acetate. The tablets can also consist of several layers.

Coated tablets can accordingly be produced by coating cores, which are produced analogously to the tablets, with agents that are commonly used in tablet coatings, for example, polyvinylpyrrolidone or shellac, gum arabic, talc, titanium oxide or sugar. In this case, the shells for the coated tablets can also consist of several layers, whereby the adjuvants mentioned above with the tablets can be used.

Solutions or suspensions with the compounds of general formula I according to the invention can contain additional taste-improving agents such as saccharin, cyclamate or sugar, as well as, e.g., flavoring substances such as vanilla or orange extract. In addition, they can contain suspending adjuvants such as sodium carboxymethyl cellulose or preservatives such as p-hydroxybenzoate.

The capsules that contain compounds of general formula I can be produced by, for example, the compound(s) of general formula (I) being mixed with an inert vehicle such as lactose or sorbitol and encapsulated in gelatin capsules.

The prodrugs according to the invention can be synthesized according to the following examples, whereby the latter are used for a more detailed explanation without limiting the invention.

General Synthesis Instructions

Variant 1

Reaction with Disulfonic Acid Chlorides

A disulfonic acid chloride of general formula SO₂—X—SO₂Cl is dissolved under a cover gas in a base, such as, e.g., pyridine. The corresponding amount of a drug substance is added to the solution. The reaction mixture is stirred until the reaction is completed. Then, the reaction mixture is stirred into a concentrated NH₃ solution. The precipitate is filtered off, washed with water and dried. The residue is extracted with an organic solvent, such as, e,g., ethyl acetate, the organic phase is washed, and it is dried with a desiccant such as, e.g., MgSO₄. After filtration, it is concentrated by evaporation and chromatographed on silica gel. Corresponding sulfamoyl sulfonates are obtained.

Variant 2

Reaction with Sulfamoyl Sulfonic Acid Halides

A drug substance, as defined above, is dissolved under a cover gas in a base, such as, e.g., pyridine, and an inert solvent, such as, e.g., chloroform. While being cooled, the corresponding amount of a sulfamoyl sulfonic acid halide of general formula NH₂SO₂—X—SO₂Hal is added to the solution. The reaction mixture is stirred until the reaction is completed. Then, water is added, and it is optionally acidified with an acid, such as, e.g., 10% HCl. It is extracted with an organic solvent, such as, e,g., ethyl acetate, the organic phase is washed, and it is dried with a desiccant, such as, e.g., MgSO₄. After filtration, it is concentrated by evaporation and chromatographed on silica gel. Corresponding sulfamoyl sulfonates are obtained.

The corresponding sulfamoyl sulfonic acid halides or disulfonic acid chlorides are commercially available or can be produced by methods that are known to one skilled in the art.

### SYNTHESIS EXAMPLES

### Example 1

### 3-tert-Butyldimethylsilyloxyestra-1,3.5(10)-trien-17β-yl 3′-sulfamoylphenyl Sulfonate

1.9 g of 1,3-benzenedisulfonyl chloride is dissolved under argon in 5 ml of pyridine. Then, 1.0 g of 3-tert-butyldimethylsilyloxyestra-1,3,5(110)-trien-17β-ol is added. After 2 hours, the reaction mixture is stirred into 25 ml of a concentrated ammonia solution. After 10 minutes, it is suctioned off, washed with water and dried. The residue is chromatographically purified on silica gel. 3-tert-Butyldimethylsilyloxyestra-1,3,5(10)-trien-17β-yl 3′-sulfamoylphenyl sulfonate is obtained.

¹H-NMR (DMSO-D₆): 0.14 (s, 6H, SiMe), 0.77 (s, 3H, 18-Me), 0.93 (s, 9H, t.-Bu), 4.43 (t, 1H, 17-H), 7.68 (s, 2H, NH₂).

### Example 2

### 3-Hydroxyestra-1,3,5(10)-trien-17β-yl 3′-sulfamoylphenyl Sulfonate

300 mg of 3-tert-butyldimethylsilyloxyestra-1,3,5(10)-trien-17β-yl 3′-sulfamoylphenyl sulfonate is dissolved in 20 ml of THF. While being stirred, 200 mg of tetrabutyl ammonium fluoride is added at room temperature. After 1 hour, 20 ml of water is stirred in. The substance is extracted with ethyl acetate. The organic phase is washed with saturated NaCl solution, dried on MgSO₄, filtered, concentrated by evaporation and chromatographed on silica gel. 3-Hydroxyestra-1,3,5(10)-trien-17β-yl 3′-sulfamoylphenyl sulfonate is obtained.

¹H-NMR (DMSO-d₆): 0.76 (s, 3H, 18-Me), 4.43 (t, 1H, 17-H), 7.68 (s, 2H, NH₂), 8.98 (s, 1H, 3-OH).

### Example 3

### 3-Oxo-7α-methylestra-4-en-17β-yl 3′-sulfamoylphenyl Sulfonate

1.9 g of 1,3-benzenedisulfonyl chloride is dissolved under argon in 5 ml of pyridine. Then, 1.0 g of MENT is added. After 2 hours, the reaction mixture is stirred into 25 ml of concentrated ammonia solution. After 10 minutes, it is suctioned off, washed with water and dried. The residue is chromatographically purified on silica gel. 3-Oxo-7α-methylestr-4-en-17β-yl 3′-sulfamoylphenyl sulfonate is obtained.

¹H-NMR (DMSO-D₆): 0.66 (d, 3H, 7-Me), 4.38 (t, 1H, 17-H), 5.69 (s, 1H, 4-H), 7.67 (s, 2H, NH₂), 7.83-8.28 (m, 4H, H—Ar).

### Example 4

### 3-Oxo-androst-4-en-17β-yl 3′-sulfamoylphenyl Sulfonate

Variant 1

2.0 g of 1,3-benzenedisulfonyl chloride is dissolved under argon in 5.5 ml of pyridine. Then, 1.0 g of testosterone is added. After 2 hours, the reaction mixture is stirred into 25 ml of concentrated ammonia solution. After 10 minutes, it is suctioned off, washed with water and dried. The residue is chromatographically purified on silica gel. 3-Oxo-androst-4-en-17β-yl 3′-sulfamoylphenyl sulfonate is obtained.

Variant 2

1.0 g of testosterone is dissolved under argon in 5.5 ml of pyridine. Then, 1.8 g of 3-aminosulfonylphenylsulfonyl chloride is added. After 2 hours, the reaction mixture is stirred into 25 ml of water and acidified with 10% HCl. After 10 minutes, it is suctioned off, washed with water and dried. The residue is chromatographically purified on silica gel. 3-Oxo-androst-4-en-17β-yl 3′-sulfamoylphenyl sulfonate is obtained.

¹H-NMR (DMSO-D₆): 0.78 (s, 3H, 18-Me), 1.11 (s, 3H, 19-Me), 4.34 (t, 1H, 17-H), 5.60 (s, 1H, 4-H), 7.67 (s, 2H, NH₂), 7.83-8.28 (m, 4H, H—Ar).

### Example 5

### Zidovudine-sulfamoylphenyl Sulfonate

2.0 g of 1,3-benzenedisulfonyl chloride is dissolved under argon in 5.5 ml of pyridine. Then, 1.0 g of zidovudine is added at 0° C. After 2 hours of stirring at room temperature, the reaction mixture is added to 25 ml of concentrated ammonia solution. After 10 minutes of stirring, it is evaporated to the dry state and extracted with EE. The organic phase is concentrated by evaporation and the residue is chromatographically purified on silica gel. Zidovudine-sulfamoylphenyl sulfonate is obtained.

¹H-NMR (DMSO-D₆): 1.17 (s, 3H, Me), 2.40 (m, 2H, CH₂), 4.00 (m, 1H, CH), 4.42 (m, 2H, CH₂), 6.09 (m, 1H, CH), 7.40 (s, 1H, CH), 7.66 (s, 2H, NH₂), 7.85-8.30 (3 m+s, 5H, 4-H), 11.35 (s, 1H, NH).

### Example 6

### Cinchonidine-sulfamoylphenyl Sulfonate

2.0 g of 1,3-benzenedisulfonyl chloride is dissolved under argon in 5.5 ml of pyridine. Then, 1.0 g of cinchonidine is added at 0° C. After 2 hours of stirring at room temperature, the reaction mixture is added to 25 ml of concentrated ammonia solution. After 10 minutes of stirring, it is evaporated to the dry state and extracted with EE. The organic phase is concentrated by evaporation, and the residue is chromatographically purified on silica gel. Cinchonidine-sulfamoylphenyl sulfonate is obtained.

¹H-NMR (DMSO-D₆): 4.99 (m, 2H, CH═CH₂), 5.94 (m, 1H, CH═CH₂), 7.58 (s, 2H, NH₂).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius, and all parts and percentages are by weight, unless otherwise indicated.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2005 057 408.4, filed Dec. 30, 2005, and U.S. Provisional Application Ser. No. 60/742,526, filed Dec. 6, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Sulfamoyl sulfonate prodrugs of general formula I [symbol]
[Group Z] (I)
in which
X is a C1-12-alkanediyl-, a CpF2p group where p=1-5, a C3-8-cycloalkanediyl-, an arylene-, a heteroalkanediyl-, a C1-4-alkanediylaryl-, a C1-4-alkanediyl-C3-8-cycloalkyl- or a C3-8-cycloalkanediyl-C1-4-alkyl group, and
Drug is a pharmaceutical active ingredient that can form a sulfonate with an OH group, such as steroids, anti-malaria agents, nucleosides, or isoflavonoids,
which can optionally be substituted.

2. Sulfamoyl sulfonate prodrugs according to claim 1, whereby
Drug means steroids, such as estrogens, for example estradiol or estriol, or
androgens, for example testosterone, MENT (7α-methyl-19-nortestosterone), eF-MENT (11-fluoro-7α-methyl-19-nortestosterone), nandrolone, DHT (dihydrotestosterone), or
gestagens, for example norethisterone, dienogest or levonorgestrel, corticoids, for example cortisol
anti-malaria agents, for example quinine, cinchonidine, hydroxychloroquine, primaquine, mefloquine or
nucleosides consisting of a sugar, such as ribose or deoxyribose, and a base, such as adenine, guanine, cytosine, thymine or uracil, and also zidovudine, brivudine, indinavir, nelfinavir isoflavonoids, for example genisteine.

3. Sulfamoyl sulfonate prodrugs according to claim 1, whereby X is an arylene group.

4. Sulfamoyl sulfonate prodrugs according to claim 3, whereby X is a phenylene-, pyridylene- or thiophenylene radical that is unsubstituted or that is substituted with a chlorine.

5. Sulfamoyl sulfonate prodrugs according to claim 3, namely
1) 3-hydroxyestra-1,3,5(10)-trien-17β-yl 3′-sulfamoylphenyl sulfonate,
2) 3-acetoxyestra-1,3,5(10)-trien-17β-yl 3′-sulfamoylphenyl sulfonate,
3) 3-tert-butyldimethylsilyloxyestra-1,3,5(10)-trien-17β-yl 3′-sulfamoylphenyl sulfonate,
4) 3-hydroxyestra-1,3,5(10)-trien-17β-yl 4′-sulfamoylphenyl sulfonate,
5) 2-methoxy-3-hydroxyestra-1,3,5(10)-trien-17β-yl 3′-sulfamoylphenyl sulfonate,
6) 3,16α-dihydroxyestra-1,3,5(10)-trien-17β-yl 3′-sulfamoylphenyl sulfonate,
7) 3,17β-dihydroxyestra-1,3,5(10)-trien-16α-yl 3′-sulfamoylphenyl sulfonate,
8) 3-benzoyloxyestra-1,3,5(10)-trien-17β-yl 3′-sulfamoylphenyl sulfonate,
9) quinine-3′-sulfamoylphenyl sulfonate,
10) cinchonidine-3′-sulfamoylphenyl sulfonate,
11) zidovudine-3′-sulfamoylphenyl sulfonate,
12) 3-oxoandrost-4-en-17β-yl 3′-sulfamoylphenyl sulfonate,
13) 3-oxoandrostan-17β-yl 3′-sulfamoylphenyl sulfonate,
14) 3-oxo-7α-methylandrost-4-en-17β-yl 3′-sulfamoylphenyl sulfonate,
15) 3-oxoestr-4-en-17β-yl 3′-sulfamoylphenyl sulfonate, and
16) brivudine-3′-sulfamoylphenyl sulfonate.

6. Compounds according to claim 1, whereby the active ingredient is an anti-malaria agent, such as arteether, artemether, artesunate, chloroquine, pamaquine, primaquine, pyrethamine, mefloquine, proguanil, cinchonidine, cinchonin, hydroxychloroquine, pamaquine, primaquine, pyrimethamine, quinine, or a quinine derivative, such as quinine-bisulfate, quinine-carbonate, quinine-dihydrobromide, quinine-dihydrochloride, quinine-ethylcarbonate, quinine-formate, quinine-gluconate, quinine-hydroiodide, quinine-hydrochloride, quinine salicylate or quinine-sulfate.

7. Use of the compounds according to claim 6 for the prevention of a parasitic attack of erythrocytes.

8. Compounds according to claim 1, whereby the therapeutically desired effect takes place by release, especially hydrolytic cleavage of the active ingredient contained in the prodrug or its metabolites.

9. Pharmaceutical composition containing at least one compound of general formula I according to claim 1 and optionally at least one additional active ingredient together with pharmaceutically compatible adjuvants and/or vehicles.

10. Pharmaceutical composition according to claim 9, whereby the additional active ingredient is a steroidal compound.

11. Pharmaceutical composition according to claim 10, whereby the additional steroidal compound is a gestagen, antigestagen or a progesterone receptor modulator.

12. Pharmaceutical composition according to claim 11, in which the included gestagens are norethisterone, dienogest, drospirenone, or levonorgestrel; antigestagens are mifepristone, onapristone, and progesteron receptor modulators, for example, mesoprogestins, such as asoprisnil.

13. Use of the compounds according to claim 1 for the production of a pharmaceutical agent.

14. Use according to claim 13 for the production of a pharmaceutical agent for hormone replacement therapy.

15. Use of the compounds according to claim 1 for female birth control.

16. Use according to claim 13 for the production of a pharmaceutical agent for therapy and/or prophylaxis of hormonally-induced diseases in men and women.

17. Use according to claim 13 for the production of a pharmaceutical agent for therapy and prophylaxis of endometriosis, breast cancers, prostate cancers or hypogonadism.

18. Use according to claim 13 for the production of a pharmaceutical agent for therapy and/or prophylaxis of diseases that are positively influenced by the inhibition of carbonic anhydrase activity.

19. Use according to claim 13 for the production of a pharmaceutical agent for therapy and/or prophylaxis of inflammatory and/or allergic diseases.

20. Process for the production of sulfamoyl sulfonate prodrugs of general formula (I) according to claim 1 by reaction of a corresponding active ingredient “Drug” according to claim 1, with a disulfonic acid chloride SO2-X—SO2Cl in the presence of a base, and subsequent treatment with ammonia, or by reaction of the corresponding active ingredient “Drug” according to claim 1 with a sulfamoylsulfonic acid halide NH2SO2-X—SO2Cl in the presence of a base.

21. Process according to claim 20, whereby the base is pyridine.
